(19) United States
(12) Patent Application Publication
(10) Pub. No.: US 2005/0127843 A1
(43) Pub. Date: Jun. 16, 2005

(54) Device for treating surfaces of containers with plasma

(76) Inventors: Pavel Koulik, Blaesheim (FR); Mickhail Samsonov, Illkirch-Graffenstaden (FR); Alexander Cherepanov, Illkirch-Graffenstaden (FR); Evguenii Petrov, Illkirch-Graffenstaden (FR); Oleg Borissov, Illkirch-Graffenstaden (FR); Robert Lohr, Hangenbieten (FR); Sebastien Vanaud, Strasbourg (FR); Vladimir Rung, Illkirch-Graffenstaden (FR)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 10/510,999

(22) PCT Filed: Apr. 24, 2003
(86) PCT No.: PCT/IB03/01675

(30) Foreign Application Priority Data
Apr. 24, 2002 (EP) 024053335.7

(51) Int. Cl. H01J 7/24

(52) U.S. Cl. 315/111.01; 315/111.21

(57) ABSTRACT

The invention concerns a device for treating surfaces of containers with plasma comprising a kinematic system for transporting the containers, and a plurality of plasma generators operating at atmospheric pressure, each generator being designed to treat one container at a time, the plasma generator comprising a treatment gas supply system and an electric power supply system including at least one transistor serving as switch, or an LC adapter, designed to supply pulses to the current.

The present invention relates to a device for treating surfaces of containers with a plasma, for example for waterproofing and sterilising bottles made of a plastic material, at industrial production rates.

The treatment of surfaces of containers with a plasma is already known and has been proposed or used industrially by a number of firms, such as the firms SIDEL, TETRAPAK and KRONES. The devices proposed or used industrially by these firms, make use of plasmas generated by microwave generators or high-frequency (HF) generators, under vacuum. These devices and methods are described in a number of publications, for example:
(1) the “ACTIS” system, proposed and used industrially by the firm SIDEL, in SIDEL News, le journal des clients, September 2001, p. 89, 9.
(2) the “GLASKIN” system, proposed and used industrially by the firm TETRAPAK, in Tetrapak Business Area Plastics, September 2001,
(3) the BEST PET system, proposed and used industrially by the firm KRONES, in Krones News, September 2001.

These devices suffer the drawback that the generation of the plasma is carried out under vacuum and, as a result, they necessitate an equipment including vacuum pumps and air-tight conduits. This makes these installations expensive, lacking versatility, very cumbersome and difficult to integrate into industrial lines for filling PET bottles with beverages (beer, mineral waters, carbonated beverages, milk and milk products).

In the case of the equipment proposed by the firm SIDEL, in which the bottle is treated on a carrousel type of installation, the installation must be equipped with friction seals, which are not very reliable and where a strictly reproducible vacuum from one bottle to the other is difficult to achieve.

Furthermore, the treatment cycle of the container necessitates a step of putting the container under vacuum, which in practice constitutes a loss of time in the treatment process.

The methods for treating surfaces of containers with an atmospheric plasma such as those described in the international patent application PCT/IB02/01001 make it possible to avoid the above cited drawbacks. However, in these prior art methods or in said international patent application, a plasma treatment, to be efficient, would necessitate a duration of treatment which would be well above that needed for maintaining a proper production rate in the industrial installations used for filling containers.

For example, in the case of the impermeability treatment of PE bottles with an atmospheric plasma according to the method described in the international patent application PCT/IB02/01001, the duration of the treatment is in the order of 30 seconds, while an installation producing and filling the bottles operates at a production rate of up to 40 000 bottles per hour, which corresponds approximately to 10 bottles per second. In order to satisfy the requirements relating to industrial production rates, 300 bottles would need to be treated at the same time to ensure a residence time of 0.1 second per bottle. The treatment devices, if they were to be used industrially, would thus need to provide for an accumulation of containers, treatment of the containers carried out in parallel, and distribution of the containers after their treatment. The number of containers treated in parallel is equal to the product of the throughput (productivity) by the duration of the treatment of each container. For instance, if the industrial throughput is of 10 bottles per second and the duration of the treatment 30 seconds, the number of containers treated in parallel should be 300.

In view of the above, an object of the invention is to provide a device for treating the surface of containers, such as bottles, with an atmospheric plasma at industrial production rates.

It is advantageous to provide a device for treating the surface of bottles, which can be easily incorporated in an industrial bottle production and filling line and which is compact, reliable and cost effective.

It is advantageous to provide a device for treating surfaces with a plasma, including a plasma generator operating under atmospheric pressure and generating a plasma enabling a high quality treatment of the inner surface of bottles and, in particular the sterilisation and the deposition of a barrier film, for example inside PET bottles.

The objects of the invention are achieved with a device for treating the surface of containers with an atmospheric plasma according to claim 1.

In the present invention, a device for treating the surface of containers with a plasma comprises a kinematic system for the transport of the containers and a plurality of plasma generators operating at atmospheric pressure, each plasma generator being designed for treating one container at a time and comprising a system for supplying a treatment gas and a system for supplying a current, comprising at least one transistor acting as a switch, or an LC adapter, to supply the current in pulses. Each generator is advantageously provided as a column of a diameter or of a width close to the diameter or to the width of the container, or slightly broader.

In the power supply system of the generators comprising LC adapters, the current pulses are generated by a central (common) current supply and distributed through conductive lines, for example coaxial lines, to a plurality of plasma generators functioning in parallel. The LC adapter of each generator makes it possible to adjust the power absorbed by the discharge (plasma) to that generated by the central current supply, i. e. to adapt the impedance of the load to that of the source.

In the electrical power supply system of the generators comprising interrupter transistors, the current pulses for the discharge are generated or controlled individually in each generator, these generators being accordingly capable of being connected to an electrical power network or some other external source of electric energy, without necessitating the special measures required when using a central power supply distributing power to generators arranged in parallel.

The electrical power supply system of the generators can comprise or be connected to a control unit designed for controlling the magnitude and the slope of the leading edge of the electric current pulses, their duration and their frequency. The duration and the frequency of repetition of the pulses are, accordingly, adjusted and controlled with a transistor generator of a small bulk and of a low cost. In the LC adapter version, the adapters have also a small bulk and their cost is low. This makes it possible to provide the treatment device with a plurality of plasma generators, in order to carry out the treatment on a plurality of parallelly placed containers, with each container being treated by an individual generator.

The use of the generators according to the invention makes it possible to generate plasmas at atmospheric pressure, under conditions which are shifted away from the state of thermodynamic and chemical equilibrium and the adjustment and the control of the characteristic features of the pulses and their frequency, in particular the time elapsed between two pulses, makes it possible to vary the chemical activity of the excited particles, atoms, molecules, radicals and clusters of the plasma, in order to achieve a treatment of the quality desired.

The generators of electric current pulses, of small dimensions, can function under forced state conditions, i. e. when the elements of the generator are forced to generate at an energy regime exceeding their capacity, but in a non-stationary thermal state. Under forced state conditions, a cooling of elements of the generator between the pulses may prove necessary and, to this end, the pulses can be spaced apart by relatively long interruptions, to enable a cooling before the following pulse. The heat transfer regime of these pulse generators is thus in a non-steady state.

The different embodiments of the kinematic systems according the invention, which will be described hereafter in relation with the figures, make it possible to ensure a transport of the containers which is rapid and a positioning thereof which is precise and reliable, beneath the corresponding plasma generators.

Other objects and advantageous features of the invention will become apparent from the claims, from the description of embodiments of the invention made hereafter and from the drawings, in which:

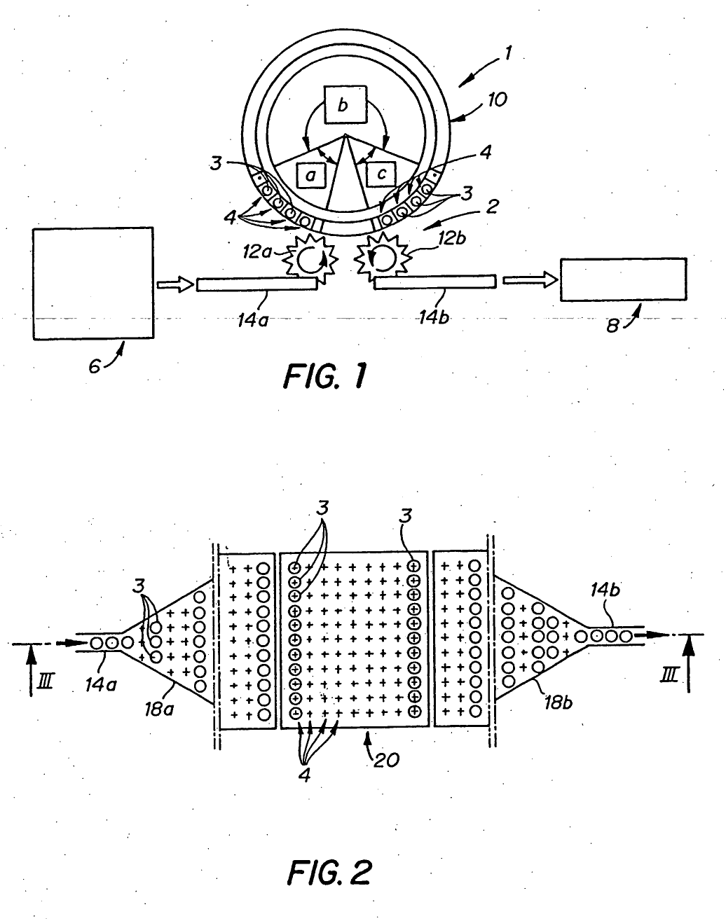

FIG. 1 is a simplified plan view of a device for treating the surface of containers, in particular of bottles, by the use of an atmospheric plasma, according to the invention, illustrating, in particular, the kinematic system;

FIG. 2 is a simplified plan view of another embodiment of a device for treating the surface of bottles, by the use of an atmospheric plasma, according to the invention, illustrating in particular the kinetic system;

FIG. 3 is a simplified cross-sectional view, taken along lines III-III of FIG. 2, of a device for treating the surface of bottles, by the use of an atmospheric plasma, according to the invention;

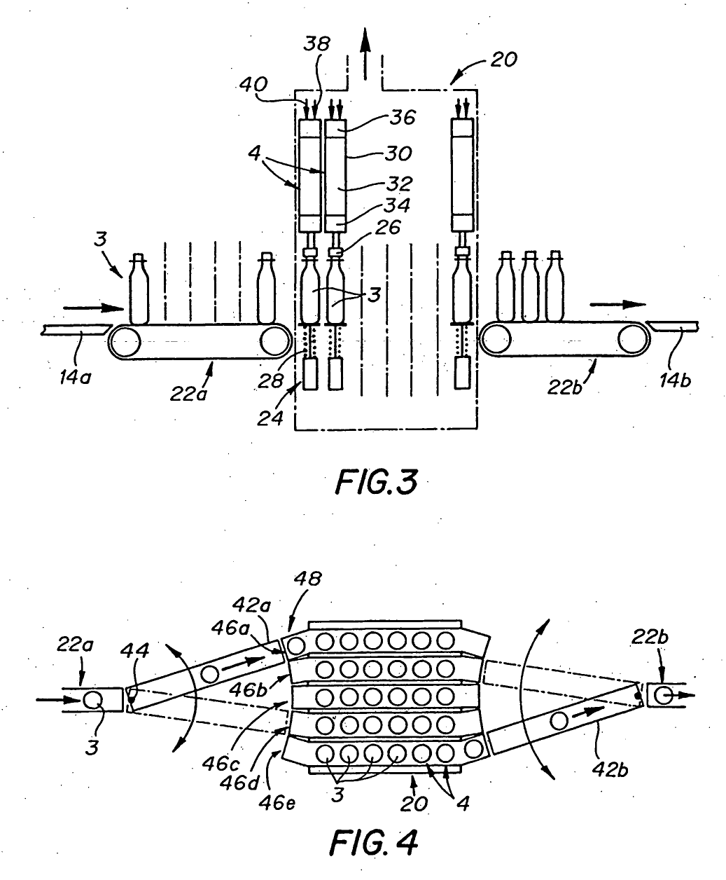

FIG. 4 is a simplified plan view of another embodiment of a device for treating the surface of bottles, by the use of an atmospheric plasma, according to the invention, illustrating in particular the kinematic system;

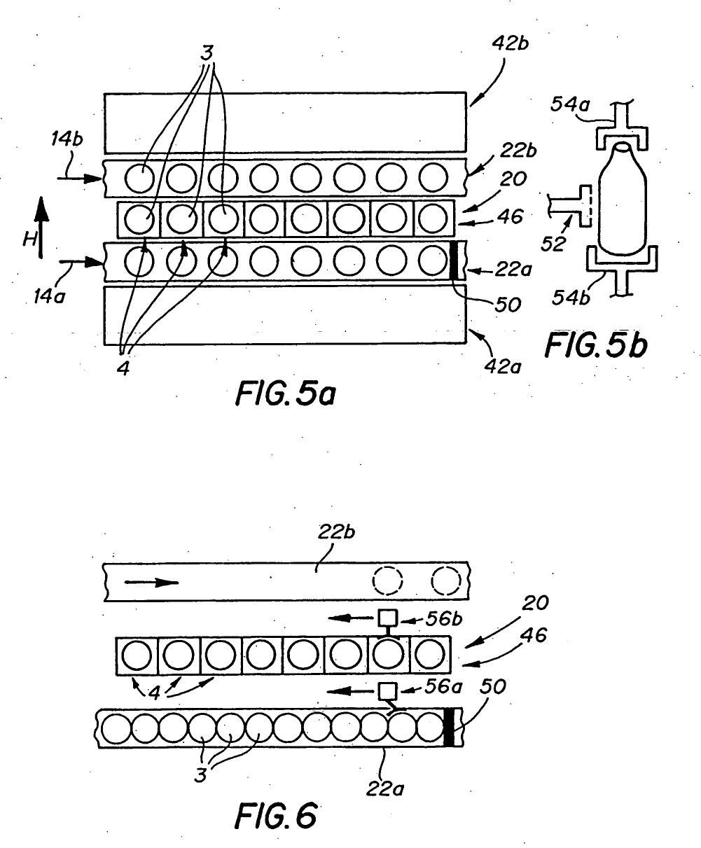

FIG. 5a is a simplified plan view of another embodiment of a device for treating the surface of bottles, by the use of an atmospheric plasma, according to the invention, illustrating more particularly the kinematic system;

FIG. 5b is a side view of a portion of the device of FIG. 5a;

FIG. 6 is a simplified plan view of another embodiment of a device for treating the surface of bottles, by the use of an atmospheric plasma, according to the invention, illustrating more particularly the kinematic system;

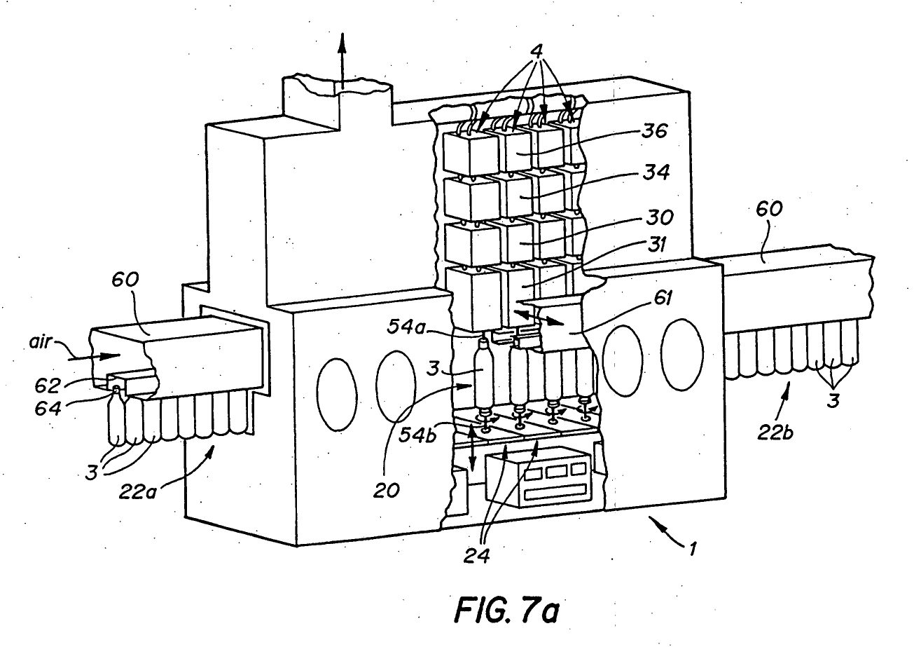

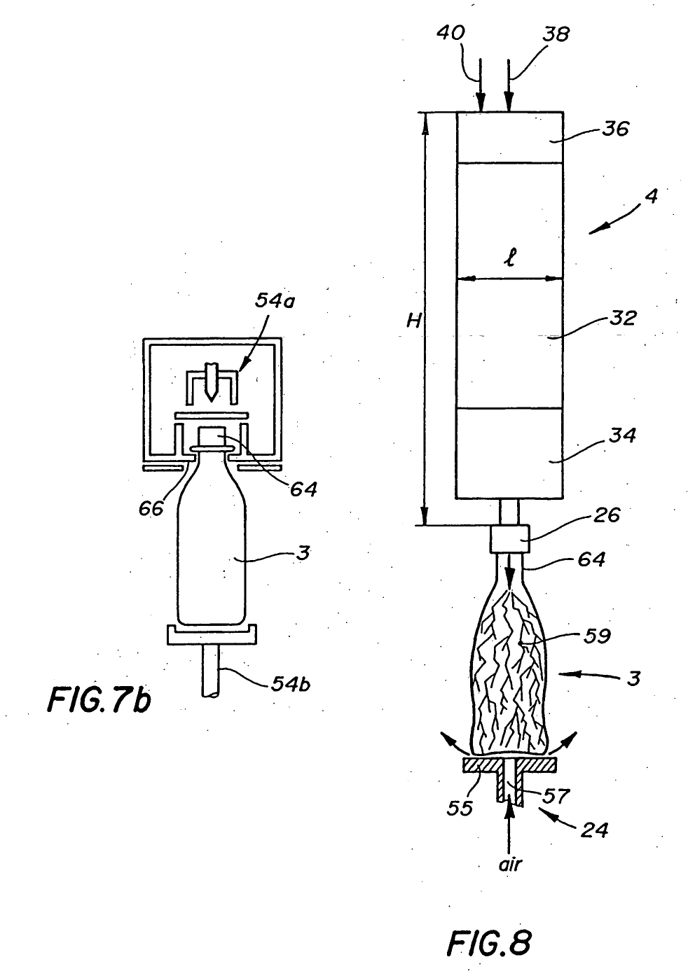

FIG. 7a shows a simplified perspective view of another embodiment of a device for treating the surface of bottles, by the use of an atmospheric plasma, according to the invention;

FIG. 7b is a simplified cross-sectional view of a portion of a plasma generator and of a bottle, during the treatment;

FIG. 8 is a simplified view of an embodiment of a plasma generator and of a bottle, during the treatment, according to the invention;

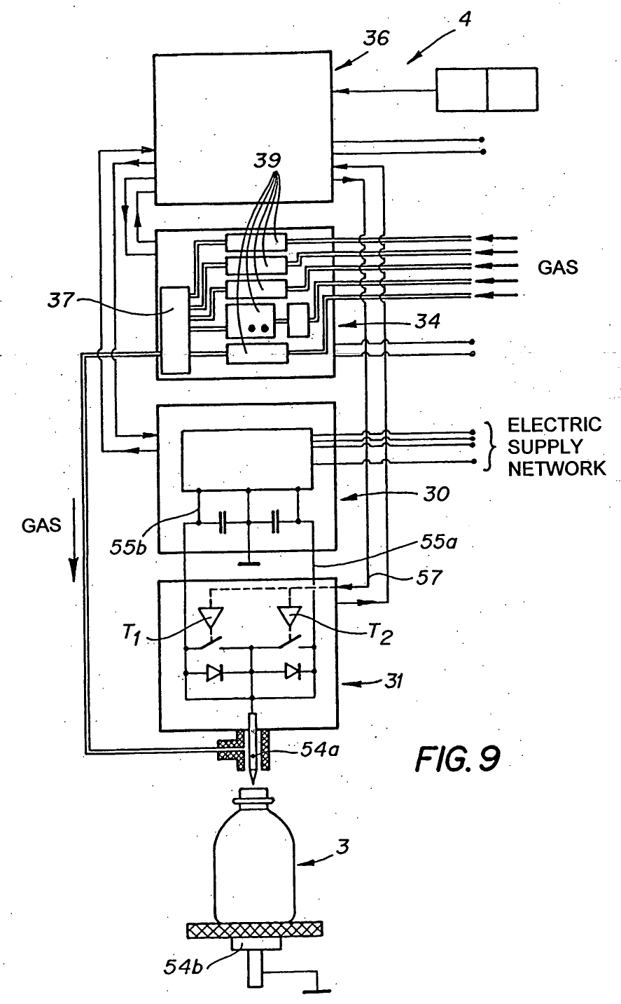

FIG. 9 is a functional representation of another embodiment of a transistor plasma generator of the device according to the invention;

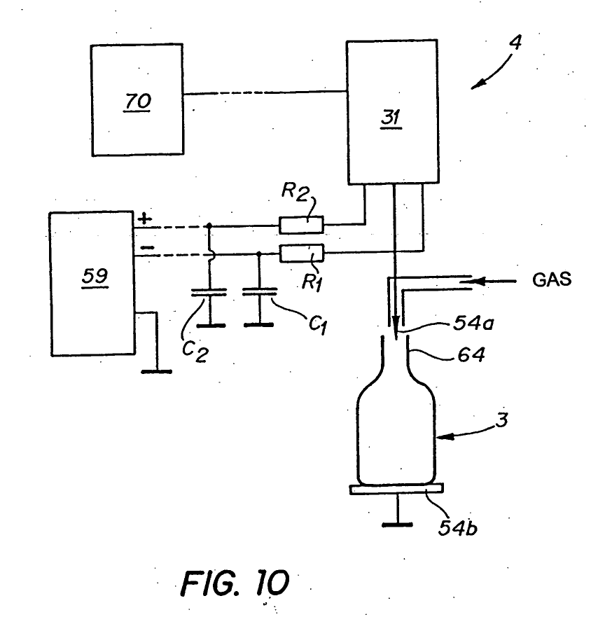

FIG. 10 is a simplified view illustrating the electrical power supply of a transistor generator according to another version of the invention;

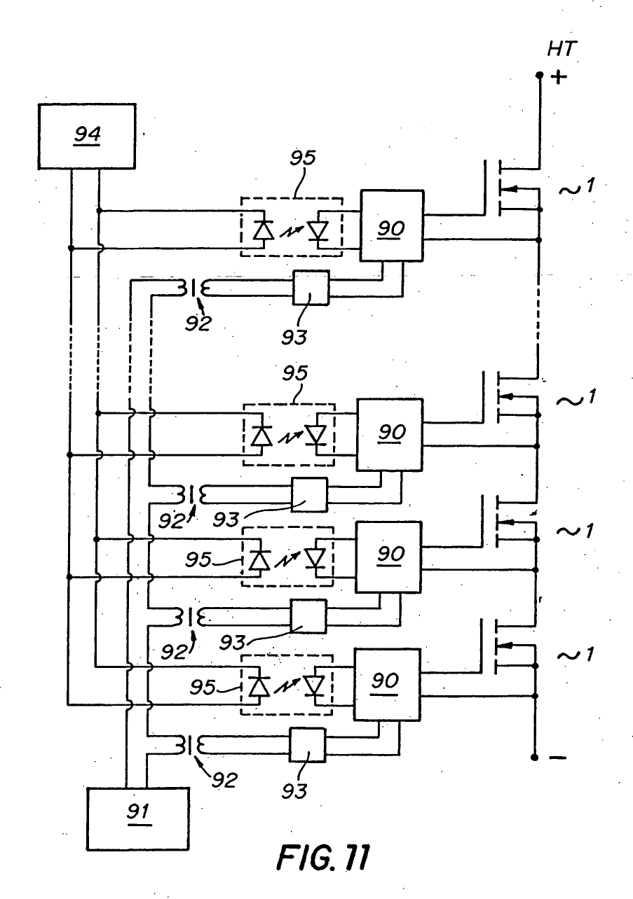

FIG. 11 is a circuit diagram of an electrical power supply with a high voltage optical separator of a generator according to another version of the invention;

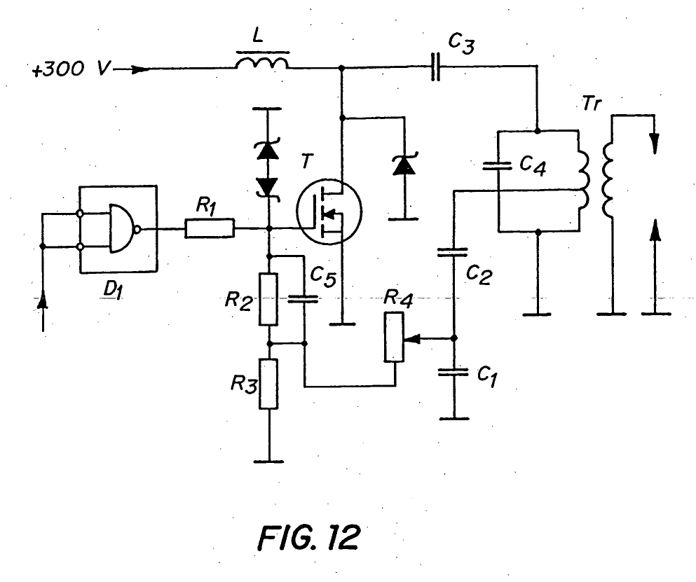

FIG. 12 is a circuit diagram of an electrical power supply of a generator, based on the use of a field transistor, according to another version of the invention;

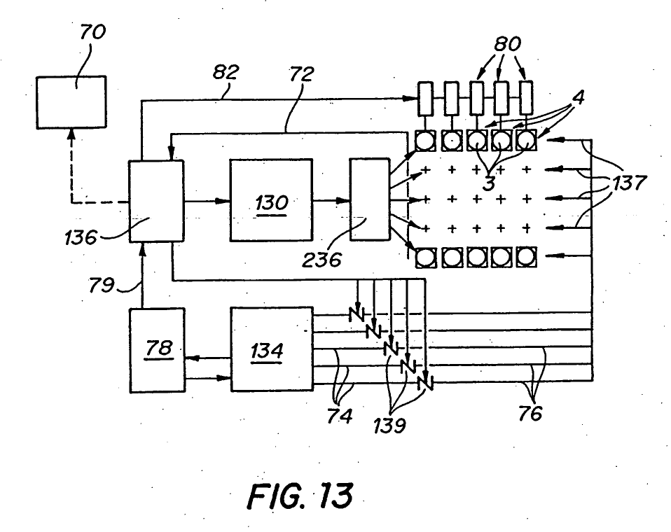

FIG. 13 is a schematic view of an embodiment of a device according to the invention with an electrical power supply, power supplying a plurality of plasma generators from a central power supply block; and

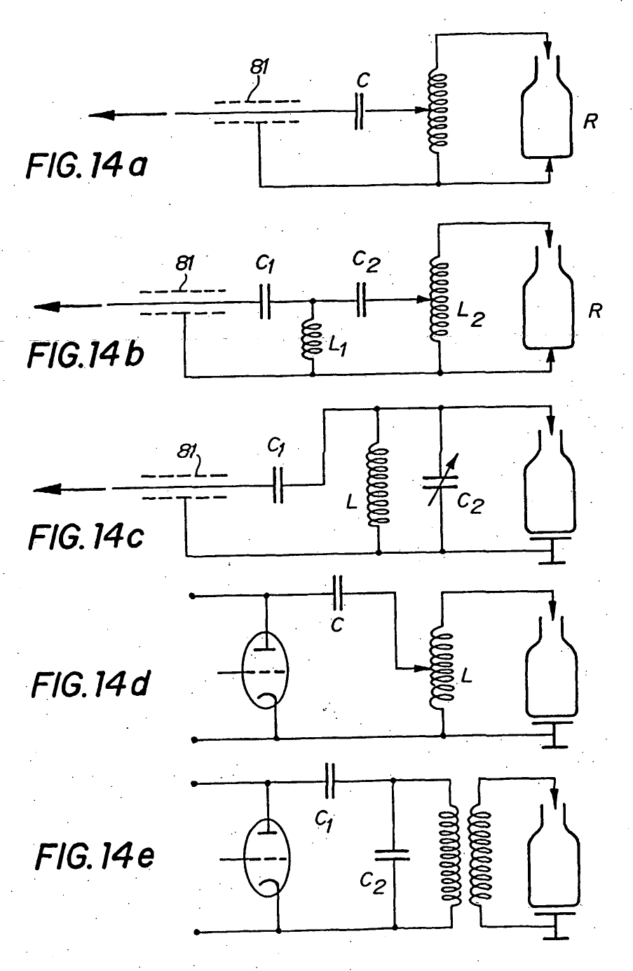

FIGS. 14a to 14e are circuit diagrams of different versions of the electrical power supply of plasma generators with an LC adapter, in the case where a plurality of generators are connected to a central power supply block, such as the one illustrated in FIG. 13.

Referring to the figures and, more particularly, to FIGS. 1 to 8, a device for the treatment with an atmospheric plasma of the surface of containers—for example for treatments such as the sterilisation of the inner surfaces of PET bottles and the deposition of barrier films thereupon—includes a kinematic system 2 for the transport and the positioning of the containers during treatment and a plurality of plasma generators 4. Each plasma generator 4 is designed for carrying out a full treatment cycle on a single container at a time (for example cleaning, activation, deposition of a film and sterilisation) and the plurality of generators arranged in or along the kinematic system make it possible to treat simultaneously a plurality of containers. Each generator comprises a treatment gas supply system and an electrical power supply system for producing the discharges (plasma).

In the device according to the invention, as many plasma generators as there are bottles to be treated in parallel are thus provided in order to be able to match the productivity of an industrial line for producing and filling the containers. For example, if the treatment cycle of the surface of a bottle by an atmospheric plasma lasts 30 seconds and the production rate of the industrial line is of 10 bottles per second, one should carry out the plasma treatment on 300 bottles in parallel. In order to be compatible with usual industrial production rates, the plasma treatment devices should, for example, treat simultaneously between 10 and 100 bottles. To this end, several devices can be arranged in parallel to increase the number of simultaneous treatment operations. Furthermore, the use of a plurality of treatment devices makes it possible to guarantee that the production rate is maintained, in the case of a breakdown occurring in one device and/or when maintenance operations need to be carried out.

An the important advantage of the device according to the invention is that the plasma generators operating at atmospheric pressure, according to the invention, have a small bulk and are of a relatively simple construction, which makes possible their incorporation into a kinematic system which is relatively compact, in view of carrying out the treatment in parallel of a high number of containers. Furthermore, in a preferred embodiment, the generators are constructed in such a manner as to generate plasmas, by high frequency or unipolar pulses, with a very steep leading edge, so as to satisfy, for example, the conditions set out in the international application PCT/IB02/01001. These conditions ensure a very good treatment of the surface of containers at atmospheric pressure, avoiding, amongst other, the problems associated with plasma treatments carried out under partial vacuum.

As will be described more in detail hereafter in relation with the different embodiments, the two main types of generators—which can be miniaturised and which are capable of generating the required plasma pulses—are either generators with individual power supplies controlled by control units provided in each generator and including an interrupter transistor for producing pulses, or plasma generators power supplied in parallel from a single power supply block, with each generator including an LC adapter for adapting the impedance of the discharge to that of the high frequency and high voltage power supply source.

The plasma generators can be included and used in different kinematic systems, which will now be described, in relation with FIGS. 1 to 8.

Referring to FIG. 1, the device 1 for treating containers 3 comprises a kinematic system 2 according to a first embodiment, arranged between a station 6 in which the bottles are produced or loaded and a filling station 8 for the bottles. The kinematic system 2 comprises a carrousel 10, on which is mounted a plurality of plasma generators 4, beneath which and from which the containers 3 are, respectively placed and removed by star wheels 12a, 12b, respectively from and to conveyors 14a, 14b. In the device according to FIG. 1, the treatment of the container is carried out during the travel of the container (for instance a bottle) on the carrousel. The containers are fed either from a pallet or from a blow-molding machine used for producing, for example, PET or glass bottles 3, via the conveyor 14a and the star wheel 12a. As soon as the bottle is held in position on the carrousel, beneath a plasma generator 4, the treatment is initiated. One can distinguish three different treatment sectors: the sector (a), in which the air contained in the container is flushed out by a stream of argon or nitrogen ; the sector (b), in which the container is treated (deposition of a barrier film) ; and the sector (c), in which the residual gases are flushed out of the container by a stream of air. The container is discharged from the carrousel by means of the star wheel 12b and the conveyor 14b carries the bottle to the filling station 8. Since not only a treatment aimed at depositing a barrier film is carried out but also and simultaneously a sterilising treatment, the distance travelled to the filling station 8 must be minimised in order to limit the risk of re-contaminating the container. Conventional measures can be taken to guarantee that the containers are not re-contaminated and that they remain aseptic.

The device can also comprise a ventilation system 15, which functions to flush out any residual gases and to cool the container.

The plasma generators each comprise a system for feeding gas and a system for supplying electrical power, including or connected to a process control unit, which can be constructed as described in relation with one of the embodiments of FIGS. 8 to 14e. Further, each generator can comprise or be associated with a mechanism for rotating the container.

The treatment of containers, in the case of FIG. 1, is carried out during the motion of the container carried by the carrousel. In this case, the carrier gas is supplied via friction joints and the electrical power is supplied via an electrical friction contact (not illustrated in FIG. 1). The device for rotating the container can, however, be absent if measures are taken to ensure a uniform treatment of the entire surface of the container, such as those suggested in some of the embodiments described in the international application PCT/IB02/01001.

FIG. 2 illustrates another embodiment of the plasma treatment device 1, in which the treatment is carried out on a group of containers in “Batch”. In this case, the containers 3 are treated while they are at rest. There is then no need to provide friction joints and electrical friction contacts. The containers fed from a conveyor 14a accumulate in an accumulation chamber 18 and are transferred therefrom to a treatment zone 20, in which they are treated simultaneously by a plurality of plasma generators 4, for each bottle in the treatment zone. Thereafter, the containers enter a distribution chamber 22 and return to the conveyor. This arrangement is advantageous since it necessitates no elements for ensuring an electric contact by friction.

FIG. 3 is a cross-sectional view of the device of FIG. 2. The containers, for example PET bottles, are fed from a production line via the conveyor 14a. They are received in the accumulation chamber 18 (not illustrated in FIG. 3) and are fed by means of a conveyor 22a to the treatment zone 20, where they are mounted on a rotational mechanism 24 beneath one of the plasma generators 4 comprising a cap 26, which is contacted with the mouth of the container, the contact pressure being provided, for example, by a spring 28. The treatment gas which may be a gaseous mixture, is fed into the cap, via a tubular electrode (not illustrated in FIG. 3, but described in more detail in relation to FIGS. 8 to 14e). The electrode ensures the passage of a current into the container to be treated from an electrical power supply system 30 located in the body 32 of the generator. In addition to the electrical power supply system 30, the generator comprises a gas feeder system provided as a gas distributor 34, mainly comprised of tubes, of electro-valves and of a vaporiser (not illustrated in FIG. 3). The source of electrical power and the gas distributor are controlled by a control unit or a microcontroller 36, also mounted in the body of the generator. Inlets 38 for the gas and inputs 40 for the electrical power are provided in the upper part of the generator. The rotational mechanism 24 can include an electric motor ensuring the rotation of the container during its treatment.

A ventilation system for the containers can also be provided in the device, for ensuring the evacuation of any residual gases and the cooling of the containers during their treatment.

After the treatment, the containers are retrieved on a conveyor 22b and fed, via an exit chamber 18b (see FIG. 2) to a conveyor 14b in the direction of the filling station.

FIG. 4 illustrates a version of the present invention, in which the treatment zone 20 is fed with containers 3 by means of a loading mechanism 42a of the kinematic system, including a guide means pivoting about the axis 44.

In this case, the treatment is carried out successively on the rows 46a to 46b. As soon as one row of the kinematic system is loaded with containers 3, the plasma surface treatment is initiated. While the treatment proceeds on the containers on this row, the other rows are loaded. As soon as a row is treated, it is discharged by a discharge mechanism 42b similar to the one used for the loading operation. Two transition zones 48 ensure an accurate and a smooth motion of the containers. This device offers the advantage, by comparison with that of FIG. 3, of not including an accumulation chamber and an exit chamber for the containers. Accordingly, its bulk is small by comparison with the systems of FIGS. 1 and 2.

FIG. 5 shows a device for treating the surface of containers by the use of a plasma, according to the invention, and comprising a kinematic system ensuring, simultaneously, the loading and the unloading of rows of bottles. In this case, one can use plasma generators such as those shown in FIGS. 8 and 9. The systems supplying the water (or some other coolant), the treatment gas and the electrical power are located above and beneath the row 46 being treated. Two conveyors 22a and 22b are mounted in parallel with respect to this row 46 and alongside these conveyors a loading device and an unloading device 42a and 42b are provided which ensure the simultaneous positioning of the bottles 3 arriving from the conveyor 14a in the treatment zone 20 and their unloading, from the treatment zone to the discharge conveyor 22b.

The installation functions as follows : the bottles arrive closely packed together via the conveyor 14a. They are arrested by a stopper means 50 and separated from one another by a pneumatic system 52, to be transported together in a horizontal direction H as a row 46 into the treatment zone 20. Here, the bottles are taken from beneath and from above between the electrodes 54a and 54b. The suction unit 52 releases the containers and returns to its initial position 42a. The plasma treatment of the inner surfaces of the containers is then initiated. During this time, the operations described above are repeated on the loading conveyor, so that as soon as possible and without any loss of time, the following bottle may be fed to the treatment zone.

The discharge of the treated bottles from the treatment zone proceeds after the end of the treatment. At this time, a suction unit moves towards the bottle from the side of the discharge mechanism, the bottles are released from the electrodes 54a, 54b and the suction units move the bottles in the direction of the discharge mechanism 22b. The suction units release the bottles, which leave the treatment device in the direction of the filling station

For example, if K=10 800 bottles are to be treated per hour, namely 3 bottles per second and if the treatment time is, for example, of T=30 seconds;
τ, the time needed for replacing the bottles (loading+discharging), is for example of 3 seconds;
d, the diameter of the bottle is, for example of 0.06 m; and
l, the length of the space necessary for each bottle, is, for example of 0.1 m/bottle,
then, the number of spaces in the installation is:
   N=(T+τ)·K and in this example
   N=(30+3)·3˜200
   the length of the installation is:
   L=N·l and in this example
   L=100.0·0.1=10 m
   the width of the installation is:
   B=2a+b+2c
   wherein a is the width of the zones 4 and 5 (˜0.3 m)
b is the width of the zone 1
c is the width of the conveyor (˜0.1 m)
In this example B˜0.9 m

The speed of the bottles along the conveyor should not be lower than:
W=N·d/T+τ=K·d

In this example W=0.18 m/sec

The time needed for separating the bottles on the feeder conveyor does not determine the productivity of the installation, because it is included in the treatment time. However, its magnitude must satisfy the relation: td≦T−L/W

In case of need, one can vary the speed W. The advantage of this configuration is that the installation has a small bulk and can be installed on a line conveying bottles.

A drawback of the device described hereabove is that the mechanisms for separating and for moving the bottles are bulky (≧10 m). Furthermore, PET bottles are very light and when undergoing a transversal motion, they can loose their balance.

FIG. 6 is a schematic diagram of a linear installation with a series loading and a series discharging of the bottles 3.

The plasma generators are aligned (row 46) and the loading and discharging conveyors 22a, 22b are arranged on each side of this row. The positioning of the bottles in the treatment zone 20 corresponding to the row 46 and their discharge from this zone are carried out individually and in series, by handling robots 56.

The installation functions as follows : the bottles 3 arrive continually from the loading conveyor 22a on which they are packed one against the other on abutting against the stopper means 50. The loading robot 56a moves parallelly to the row 46, for example in the direction from the right to the left, takes hold through its suction unit of a bottle on the conveyor and places it into the closest treatment position (the space made free on the loading conveyor 22a is immediately taken up by another bottle). Then, the robot rotates by 180° and positions the bottle accurately in the treatment zone. The upper and the lower electrodes grip the bottle, the suction unit is released and the robot moves to carry out the same operation so as to fill the adjoining treatment position beneath a plasma generator 4. When the robot reaches the end of the row 46 in one direction, it returns rapidly to its initial position and starts over again the process described above.

The discharge of the bottles is carried out symmetrically by the robot 56b which takes hold of the treated bottles through its suction unit and places the treated bottles on the discharge conveyor 22b.

When the robot arrives to the end of the line, it returns rapidly to its initial position. The bottles are evacuated by a fast conveyor of the kinematic system, for example by a pneumatic conveyor.

A condition which must be satisfied, if the system is to function properly is that: W . Δt≧2d−l, in which Δt is the time elapsed between the discharge of two adjoining treatment zones. In this case, the overall width of the line remains unchanged. It is advantageous to use robots which are compact, which have a high number of degrees of freedom and which operate with a high degree of precision. The advantage of this embodiment is that there is no need for a separating system for the bottles. Furthermore, the loading and the discharging devices need little space.

FIGS. 7a and 7b illustrate another embodiment of a device according to the invention, comprising a kinematic system having an air conveyor for the bottles. In this case, the treatment line coincides with the pneumatic conveyor for the bottle manufacturing and filling line. The device is thus mounted on the pneumatic conveyor of a bottle manufacturing and filling line, the treatment of the surface by a plasma is carried out in a treatment zone 20 and the loading/discharging of the bottles is carried out in the ancillary parts 22a, 22b. The main part of the plasma treatment zone comprises electrodes 54a, 54b, a plasma generator 4 positioned above each bottle 3 and mechanisms for moving and positioning the bottles in the treatment zone.

The device functions as follows:

As in the case of a conventional pneumatic conveyor, the bottles are pushed by pressurised air supplied from a duct 60 along a pneumatic transport channel 62 functioning as a support rail for the necks 64 of the bottles. A bottle counter allows the entrance of the required number of bottles, corresponding to the number of treatment station, i. e. of plasma generators 4. A precisely positioned stopper means stops the bottles at a precise position and a separating/positioning means positions the bottles beneath the respective plasma generators 4. Different separating means can be used: for example a screw conveyor or a comb with conical teeth, or further, stopper means activated one after the other by a signal from photodiodes indicating the presence of the bottles. The bottle positioning mechanism positions the bottles accurately along the axes of the electrodes 54a, 54b of the plasma generators. The walls 61 forming the pneumatic tube in the treatment zone 20 can move to provide an access to the bottles 3. The upper electrode 54a moves downward and the lower electrode 54b moves upwards. The lower electrode 54b is provided with a mechanism for rotating the bottle via friction shoes. The neck 64 of the bottle slides, during this rotation on the lower part 66 of the housing of the upper electrode. A spring mounted on the lower electrode (not illustrated) exerts the pressure necessary for ensuring, on the one hand, that no friction occurs between the bottle and the lower electrode and, on the other hand, that the friction between the housing (which is, made, for example, of Teflon) of the upper electrode and the neck of the bottle is adequate. The surface treatment by the plasma starts upon the filling of the bottle, with the gaseous mixture used as the treatment gas.

During this time, the two removable halves of the pneumatic tube 61 are moved away from each other in such a manner as to avoid any interference and any influence on the distribution of the lines of the electric field during the treatment of the bottle. The electrodes are power supplied and the deposition process of a film is initiated. After a period of time T, the process is ended and all the operations described above are carried out in the opposite direction. The bottle is flushed by a stream of air, disengaged from the electrodes and carried away by the stream of air.

The advantages of this embodiment are:
its small dimensions
its simplicity
the high speed at which the bottles are supplied and discharged
the bottles cannot fall out
the good adaptability of the installation to bottles of different volumes and shapes.

The length L of the installation depends upon the production rate N, on the duration of the treatment cycle T of each bottle and the width I of the plasma generator.

For 10 000 bottles/hour, T=30 sec and I=0.1 m, the value obtained for L is 10 m. The transverse dimension B of the installation will depend upon the number of rows of generators forming the treatment zone and in the case of one to two rows, B can amount to 0.5 m. The durations of the loading and of the discharging of the bottles are determined by the average speed of motion of the bottles, which is, for example, in the order of 10 m/sec. Accordingly, the overall duration of the loading and of the discharging does not exceed τ=2 sec.

Advantageously, the plasma generator 4 has, the general shape of a single-bloc column, such as illustrated in FIG. 8 or of a plurality of blocs, such as illustrated in FIGS. 7a or 9 and the width of the generator is close to the diameter of the bottle or to the width I of the container, or slightly higher, in order to make it possible to place in a compact manner a plurality of generators along the kinematic line, in the treatment zone. As an example, in the case of the treatment of PET bottles having a volume of 0.7 l and the shape illustrated in FIG. 8, the generator according to the invention can be constructed with a width l of about 80 mm and a height H of about 500 mm. In the example illustrated in FIG. 8, the container 3 is supported by a lower rotatory support 55 of the rotatory mechanism 24 provided with an air conduit 57 which makes is possible to hold the bottle in position and prevent the same from tilting, through the Bernoulli effect. In this version, the generator provided as a column comprises all the elements already described in relation with FIG. 3 and the same reference numerals are used. FIG. 8 also illustrates the discharge which is generated in the form of a network of branched filaments 59, as described in the application PCT/IB02/01001.

Referring to FIG. 7a, the generator comprises a plurality of blocs comprising a bloc 30 for distributing electric pulses (electrical power supply system), a bloc for distributing gas (gas supply system) and a control unit 36.

In the electrical power distribution bloc 30 illustrated in FIG. 7, the alternating current from an electrical supply network (380 V/220 V, 50 Hz) is first rectified by a rectifier 33, to obtain a high voltage direct current, with a positive polarity and a negative polarity (for example +20 kV and −20 kV). The current is then converted into pulses, by a high frequency interrupter transistor 31.

The gas distribution bloc 34 includes a manifold 37 into which are fed several gaseous components, via electrovalves 39. Vapours of organometallic compounds can also be fed to the manifold, by means of a carrier gas such as argon.

The blocs mentioned are activated and controlled by the control unit 36.

The gas supply system 34 is miniaturised and placed at a minimal distance from the container to be treated, in such a manner that the time Δ elapsed between the point of time at which the electrovalves 39 operate and the point of time of the filling of the container corresponding to the ratio of the volume V of the spaces (tubes, valves) to the flow rate of the gas be lower than the difference between the duration of the first operation (for example the flushing of the container with argon or nitrogen) and the duration of the stationary conditions (i. e. when the flow rate is stationary).

Concerning the generators which can be used with the devices and, in particular, the kinematic system of the devices described above and which satisfy the criteria of small dimensions and of reasonably low cost, three types are proposed in the framework of this invention. It should be noted that the three types proposed are capable of generating electric discharges meeting the criteria which are set out in the invention described in the international application PCT/IB02/01001 and which make it possible to carry out very effectively and at atmospheric pressure a plasma treatment of a very high quality. In this regard, the aim is in particular to generate discharges with electric pulses satisfying the advantageous criteria set out in the above-mentioned international application.

The three types of plasma generators which can be used in the present invention are, in short, the following:
1. Individual supply of discharges in each container to be treated from individual high frequency (HF) generators, by using semi-conductor keys for transforming a direct current into HF pulses and creating the electric discharges.
2. Individual supply of discharges in each container to be treated from individual generators generating a high frequency current, by using transistors, for creating the electric discharges.
3. Parallel supply of discharges with electric pulses with parameters which are substantially identical, from a central source of high frequency and high power current, supplying a plurality of plasma generators producing said discharges.

The generators, according to the invention, differ from conventional high frequency generators which use diode tubes, which are bulky and unsuitable for supplying power individually to a plurality of bottles, in that they provide a device of industrially acceptable cost and bulk. The types of generators proposed in the framework of the present invention make it possible, not only to miniaturise plasma generators, but also to create discharges with current pulses having a leading edge, a duration and a frequency satisfying the criteria set out in the application PCT/IB02/01001.

Let us first examine the generators of the first type mentioned above.

In the generators of the first type, a high frequency field transistor is used according to an external excitation scheme, which ensures the stability of the frequency, when the load (plasma discharge) is dynamic.

In conventional generator schemes, due to the undesirable effects of the parasitic capacitance of the transistor (Miller's capacitance effect), the time in the working range is increased and, for this reason, the working frequency of such a generator does not exceed 150-200 kHz.

Referring to FIG. 12, to obtain a frequency between 1 and 100 MHz, the present invention differs from known arrangements in that the generator proposed includes a field transistor in which the influence of the “Miller” capacitance is compensated by a C₁C₂C₅R₂R₃R₄ circuit. This compensation relies on the transmission of a portion of the voltage output from the transformer T_{M} via the (amplitude-phase) circuit mentioned, to the gate of the transistor T where it is added in phase to the control voltage, to increase the second derivative of the current recharging the capacitance of the gate at the beginning and at the end of the input pulse and to condition the avalanche state of the transistor. Under such conditions, the switching time of the transistor is substantially reduced and the frequency is increased, so that the requirement for the production of a plasma described in the document PCT/1B02/01001, and in particular those relating to a duration of the leading edge of the pulses lesser than 1 μsec, are satisfied.

One can use this principle when the field transistors are parallel mounted, such as illustrated in FIG. 11: in this case, their number is determined by the output power desired.

In order to ensure a stable functioning of the generator and prevents variation in the frequency arising, for example, from the heating of certain of the elements of the generator, one can use a conventional system for automatically adjusting the frequency.

### EXAMPLE 1.1

A generator was made for producing an atmospheric plasma discharge in the form of a network of filaments in the PET bottles. For each bottle, the parameters of the generator were:
Frequency of the generator: 880 kHz
Power output per pulse: 6 kW
Dimensions:diameter: 70 mm height: 400 mm
Power supply: 300 V (direct current)

The circuit makes use of 6 parallel-mounted transistors of the 2SK2611 type and of a driver TLP250 (manufactured by Toshiba) (D₁ in FIG. 12).

The high frequency generators of the second type, as illustrated in FIG. 9, are based on high voltage and high speed switching transistor, which differ from conventional generators by their small dimensions, by the absence of diodes and of resonance contours.

The high voltage/high frequency is obtained by the connection of the discharge electrode to the positive pole and to the negative pole of a high voltage source, for example, of a direct current.

The frequency of the connections and the modulation of the voltage are controlled by a computer. One can obtain a frequency between 1 and 100 MHz.

The interrupter transistor (or the switching transistor) 31 is situated directly above the container to be treated, for example a PET bottle.

The interrupter transistor is connected to an outer bipolar source of high voltage and to a control unit 36 connected to a computer.

When using a bridge schema based on two transistors T₁, T₂, one can use an external source of unipolar high voltage current.

The high speed and high voltage interrupter transistor 31 is connected, on the one hand to an electrode 54a from which the current enters the container 3 (for example, a PET bottle) by two terminals 55a, 55b connected to the poles of an outer bipolar source of a high voltage current; and, on the other hand, via a terminal 57 to the control unit 36.

### EXAMPLE 2.1

Referring to FIG. 10, a generator for producing a pulsed discharge in the form of a branched network inside a PET bottle was constructed using a high voltage/high speed interrupter transistor 31 of the HTS 301-03-GSM type (manufactured by the firm Behlke).

The interrupter was power supplied from a high voltage bipolar source 59 (−12 kV,

+12 kV, 25 kW) via an RC circuit (R₁, C₁, R₁, C₂).

The tubular metal electrode 54a was located in the vicinity of the neck 64 of a plastic container 3. The generator gas containing hexamethyidisiloxane vapours was introduced via the electrode. The frequency of the alternating high voltage and its modulation were determined by the control pulses issued by a computer 70. A discharge in the form of a branched network was generated in this manner along the inner surface of the container. An impervious SiOX film was formed. The BIF (barrier improvement factor) for oxygen was 60.

The interrupter transistor 31 used is based on high voltage field transistors as shown in FIG. 11 and series connected to and power supplied from a source of high voltage current. This device differs from conventional devices in that a high voltage opto-electronic separator is used, which makes it possible to transmit pulses with a leading edge in the order of a nanosecond.

The interrupter functions as follows:

The switching on and off of the transistors is carried out by means of a driver 90, power supplied from the source 91 (˜50 kHz) on the transformers 92, whose primary windings are series mounted. The transformers 92 have a ferrite core and they are immersed in an insulating resin. The alternating voltage of the secondary windings is rectified by the semi-conductor bridges 93 and is transmitted to the drivers 90. The pulse generator 94 creates pulse packets, which are directed to the drivers via high voltage optical pairs 95. It is possible to send light pulses of a semi-conductor laser on photodiodes by means of optical fibres.

FIG. 13 is a schema of a device with plasma generators according to the third type mentioned above, i. e. where the distribution of the current and the mixing of the gases are carried out centrally.

The plasma treatment cycle is controlled by a central control unit 136 and is recorded by a computer 70. The computer defines the programme (software) used by a microcontroller 236. The latter controls automatically the programmed functioning of the source of current 130, via the line 72, of the gas mixer 134 via the lines 74 and 76 respectively before and after the controlled electro-valves 139, of the vaporiser 78 on the basis of the return “temperature” signal 79, as well as of the rotation motors 80, via the line 82. The gaseous mixture accedes to the set of plasma generators 4 via the manifolds 137.

The simultaneous occurrence of several electric discharges (200-300), of which the parameters have a substantially non linear behaviour in the course of time, from a single generator is difficult to implement, without special measures being taken.

In the third type of generator according to the present invention, one can use a conventional source of high-frequency current and modulate the high frequency signal (i. e. obtain pulses having a given shape and a given magnitude in the course of time) by the use of a conventional method consisting in sending appropriate signals to the gate of a triode. According to the invention, discharges in the containers are supplied via individual LC adapters such as those illustrated in FIGS. 14a to 14e provided in the plasma generator directly above the container to be treated, at a distance such that the inductive and the capacitive losses of the HF line leading to the container to be treated are substantially negligible. The diameter of the column does not exceed that of the containers, for example of a PET bottle (70 mm).

The LC adapters are connected to the generator by a coaxial cable 81. Depending on the technical requirements, one can use LC adapters with one or two contours.

### EXAMPLE 3.1

A high frequency generator (13.56 MHz) was used, having an average power of 2.4 kW and a pulse power of 42 kW. The power necessary for forming a barrier film on a PET bottle of 0.5 l was P(average)=0.4 kW and P(pulse)=7 kW. The number of discharges was 6. The number of LC adapters was 6. An adapter with one contour was used with an auto-transformation of the output inductance L. The circuit diagram of the adapter is shown in FIG. 14a.

The parameters of the adapter were:
C=250 pF; L=0.5 μH

The adjustment is carried out by varying the point of contact with the winding L (this adjustment is carried out once for a given load R, for example for a bottle.

### EXAMPLE 3.2

A high frequency generator (13.56 MHz) was used, having an average power of 40 kW and a pulse power of 700 kW. The power necessary for forming a barrier film on a PET bottle of 0.5 l was P(average)=0.4 kW and P(pulse)=7 kW. The number of loads selected (in this case PET 0.5 l bottles) was 100. The number of LC adapters was 100. The adapter device with two contours used here was characterized, by comparison with the adapter, with one contour, by a lesser influence of the load on the generator. The LC adapter is shown schematically in FIG. 14b.

The parameters of the adapter were:
C₁=250 pF; L₁=0.5 μH
C₂=85 pF; L₂=0.8 μH

The adjustment according to the load R was carried out by varying the inductances L₁ and L₂. The capacitances C₁ and C₂ were constant and had a magnitude lesser than the variable vacuum capacitances.

Such a generator with 100 adapters for 100 loads can be used for the simultaneous treatment of 100 PET bottles.

### EXAMPLE 3.3

A high frequency pulse generator was used for producing surface discharges with branched filaments. The power supply came from a high frequency generator via an adapter and differed from the existing devices in that the device used a resonant parallel circuit system, as shown in FIG. 14c.

The capacitor C₁ (=100 pF) was a separation capacitor, the capacitor C₂(=30-100 pF) was an adjustment capacitor. The inductance used was L (=0.5 AH). This device makes possible an adaptation in one step. The losses were reduced by comparison with existing systems where they can be 4 to 5 times greater than the energy used in the discharge.

### EXAMPLE 3.4

A high frequency pulse generator was used, which was a conventional high frequency generator without an adapter, and to which an independent excitation was imposed. The auto-transformation devices and the transformation devices were designed as shown in FIGS. 14d and 14e.

These solutions have the advantage of eliminating the losses occurring in the adapter and they make it possible to connect a multitude of identical loads. In this example, 4 pulsed discharges were carried out in parallel, of which the average power was 0.3 kW. The transformation coefficient was of 1.1-1.3.

1-18. (canceled)

19. A device for treating the surface of containers with a plasma, comprising a kinematic system for the transport of the containers and a plurality of plasma generators operating at atmospheric pressure, each generator adapted to treat one container at a time, the plasma generator comprising a treatment gas supply system and an electrical power supply system comprising at least one interrupter transistor functioning as an interrupter, or an LC adapter, adapted for supplying current in pulses.

20. A device according to claim 19, wherein each generator is provided as a column having a diameter or a width close to or slightly greater than the diameter or the width of a container.

21. A device according to claim 19, wherein the power supply system includes or is connected to a control unit adapted to control the amplitude of the pulses of electric current, the slope of the leading edge of these pulses, their frequency and the time elapsed between two successive pulses.

22. A device according to claim 19, wherein the plasma generators are placed side by side on a carrousel of the kinematic system.

23. A device according to claim 19, wherein the kinematic system comprises an accumulation zone for grouping the containers and in that a plurality of generators are positioned above this system for a batch treatment of containers.

24. A device according to claim 19, wherein the power supply system comprises a current source and the gas supply system comprises a gas distributor.

25. A device according to claim 24, wherein the current source, the gas distributor, and a control unit comprising a microcontroller, are controllable so as to provide a plasma treatment program for each container, individually.

26. A device according to claim 24, wherein the current source, the gas distributor and the microcontroller are provided in the same housing or as blocks above the container to be treated.

27. A device according to claim 23, wherein the kinematic system comprises a pivoting guide for directing the loading of the containers in the accumulation zone.

28. A device according to claim 19, wherein a treatment zone of the kinematic system comprises rows for storing rows of said containers in such a manner that the treatment of the containers is carried out therein row by row, as and when the rows are filled with containers.

29. A device according to claim 23, further comprising at least two compartmented complementary zones upstream and downstream of the accumulation zone, which are used for, respectively, placing the containers in rows in the accumulation zone and discharging the containers from the accumulation zone.

30. A device according to claim 19, wherein the power supply system comprises a central current source comprising a high frequency current generator producing high frequency electric pulses controlled by signals sent to a gate of a triode, the high frequency pulses being sent in parallel to each plasma generator to produce, via the LC adapters, a discharge in the form of a network of filaments in each container.

31. A device according to claim 19, wherein the power supply system comprises a central high voltage bipolar direct current source supplying individual high speed and high voltage interrupter transistors of each plasma generator.

32. A device according to claim 19, wherein the power supply system comprises a central high voltage unipolar direct current source supplying the generators, the generators being provided with bridges comprising two high speed and high voltage interrupter transistors adapted to create discharges-in the form of a “network of filaments”.

33. A device according to claim 19, wherein the power supply system comprises a central high voltage direct current source supplying the plasma generators, the generators being provided with individual field transistor systems, each having a CR amplitude-phase circuit, with the signal being modulated by a computer, each of said individual field transistor systems supplying electricity for a discharge in the form of a “network of filaments” on the inner surface of the container to be treated.

34. A device according to claim 30, wherein high power elements of a circuit of the high frequency current generator are cooled in such a manner as to function in a non-steady heat transfer state.

35. A device according to claim 19, wherein the kinematic system comprises pneumatic transport channels (62) in which the containers are moved by an air stream, the pneumatic transport channels being movable in a plasma treatment zone (20) of the device, in order to enable the access of the generator electrodes (54a) to the containers.

36. A device according to claim 21, wherein the control unit controls the execution of a program of distribution of gas portions to form the gaseous mixture constituting the treatment gas used in the plasma treatment of the containers.
